(19) United States
(12) Patent Application Publication
(10) Pub. No.: US 2017/0319832 A1
(43) Pub. Date: Nov. 9, 2017

(54) DEVICES FOR HYDRATING PATIENTS

(71) Applicant: Kelly Gardner, Elizabeth, CO (US)

(72) Inventor: Kelly Gardner, Elizabeth, CO (US)

(21) Appl. No.: 15/235,733

(22) Filed: Aug. 12, 2016

Related U.S. Application Data
(60) Provisional application No. 62/391,711, filed on May 9, 2016.

(51) Int. Cl. A61M 31/00

(52) U.S. Cl. CPC A61M 31/00

(57) ABSTRACT

A method, device, and kit for providing fluid to a patient, the device includes a food-contact-safe sponge and a handle, the handle supporting the sponge.

This application claims priority to provisional application No. 62/391,711 filed on May 9, 2016 by Kelly Gardner of Elizabeth, Colo.

### FIELD OF USE

Non-vascular hydration of patients.

### BACKGROUND

Many patients receiving medical therapies have difficulty eating and drinking normally. A variety of different technologies and approaches have been developed to assist in feeding and hydrating people. However, many of these approaches are costly, poorly tolerated, and/or use vascular or mucus membrane access which may be uncomfortable and/or a potential source of infection.

### BRIEF DESCRIPTION OF THE DRAWINGS

The accompanying drawings illustrate various examples of the principles described herein and are a part of the specification. The illustrated examples do not limit the scope of the claims. Throughout the drawings, identical reference numbers designate similar, but not necessarily identical, elements.

FIG. 1 shows a device according to an example consistent with the present disclosure.

FIG. 2 shows a device according to an example consistent with the present disclosure.

FIG. 3 is a flowchart of a method consistent with the present disclosure.

FIG. 4 shows a device according to an example consistent with the present disclosure.

### DETAILED DESCRIPTION

Chemotherapy and radiation treatment as part of cancer treatment have a number of unpleasant side effects. Many of these side effects can impact a patient's willingness and ability to remain hydrated and ingest calories. Unfortunately, in some cases, patients end up needing additional intravenous fluids and/or supplemental nutrition to endure the effects of treatment. Such interventions increase the time patients spend in a clinical setting and may negatively impact patient morale and/or outcomes.

Additionally, the salivary glands may be impacted by treatment, reducing the availability of saliva. The resulting dry mouth can have a number of negative consequences. For example, dry mouth may be uncomfortable. Dry mouth may result in local concentrate of bacteria and bacterial byproducts causing damage to teeth.

One method of addressing these issues is the use of a sponge, which is soaked in fluid and applied to the mouth. However, sponges currently used in clinical practice tend to be rough and firm due in order to adequately retain their shape after absorbing fluid. The rough and firm sponge is uncomfortable for patients and may discourage patients from getting the fluids needed to minimize the impacts of their treatment.

Accordingly, the present specification describes, among other examples, a device for providing fluid to a patient, the device comprising: a food-contact-safe sponge; and a handle, the handle supporting the sponge.

The present specification also describes a method of providing oral hydration or feeding, the method comprising: introducing a sponge on a handle into a patient's mouth, the sponge holding liquid.

The present specification also describes a kit, the kit comprising: a delivery sponge, the sponge to store liquid in an expanded state and release stored liquid upon compression and a handle to couple to the sponge, wherein handle supports the sponge and allows manipulation of the sponge.

Turning now to the drawings.

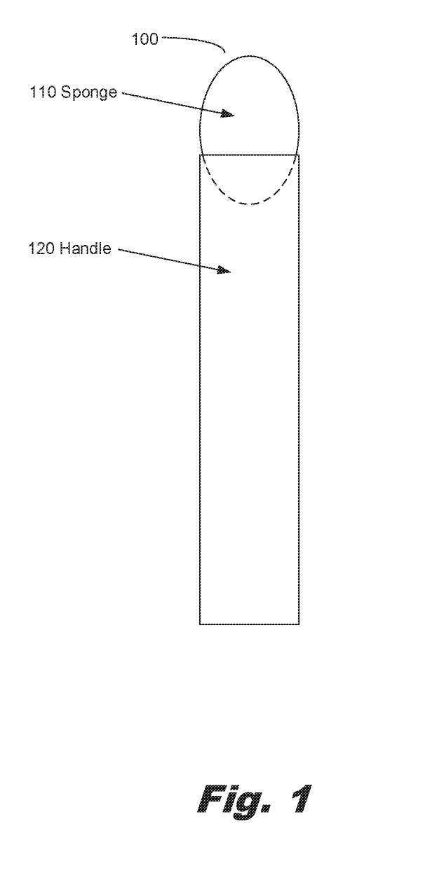

FIG. 1 shows a device according to one example consistent with this disclosure. The device (100) comprises: a sponge (110) and a handle (120) connected to the sponge (110).

The device (100) is a used for providing liquids to a patient. The handle (120) allows a user to hold and manipulate the device (100). This reduces contact between a user and the liquid which is both more sanitary and less messy. The user allows the sponge (110) to absorb the liquid. The user then inserts the sponge (110) into the patient's mouth. The patient's tongue can be used to express the liquid from the sponge (110). In another example, the sponge (110) can be pressed against the tongue, teeth, cheek, or a similar surface in the patient's mouth to express the fluid from the sponge (110).

The sponge (110) may be removable from the handle (120). The sponge (110) may be attached to the handle (120). In one example, the sponge (110) is a disposable while the handle (120) is a durable medical device. In another example, the sponge (110) and handle (120) are a disposable medical device. In a third example, the sponge (110) and handle (120) are both reusable.

In one example, the sponge (110) is oval in shape. The sponge (110) may have a principle axis of between 0.25 and 3 inches. In one example, the (110) sponge has a principle axis of approximately 1.5 inches in length. The sponge (110) may have a minor axis of between 0.25 and 2.5 inches in width. In one example, the sponge has a minor axis of approximately 0.75 inches in width. The sponge (110) may have a height of 0.1 to 2 inches. In one example, the sponge (110) has a height of approximately 1.25 inches. Other suitable geometries may be selected based on the size of the patient.

The sponge (110) may hold 4 milliliters (ml) to 15 ml of liquid. In one example, the sponge (110) holds approximately 5 ml of liquid. The sponge (110) may be selected to be easily distended and compressed. This helps prevent irritation and pain in the sensitive tissues inside the mouth. The stiffness of the sponge (110) may be selected to provide adequate recoil to allow recovery when filling the sponge (110) with liquid.

The sponge (110) is a food contact safe material. In one example, the sponge (110) complies with 21 C.F.R. §174-179. Specifically, the sponge (110) may comply with 21 C.F.R. §177, which is incorporated by reference.

The handle (120) allows manipulation of the sponge (110) without contact. In one example, the handle (120) is approximately 5 inches in length and 0.75 inches in diameter. The handle (120) may taper toward the distal end, i.e. toward the sponge (110). The handle (120) map taper toward the proximal end. The handle (120) may include an endcap on the proximal end.

The handle (120) may be formed from a food contact safe material. In one example, the handle complies with 21 C.F.R. §174-179.

The handle (120) may be molded over the sponge (110). The handle (120) may releasable attach to the sponge (110). The handle (120) may include a fastening component to secure the sponge (110). The handle (120) may include a snap cap that secures the sponge (110) to the handle (120). In one example, the ability to separate the sponge (110) from the handle (120) facilitates cleaning and reuse. In another example, the sponge (110) and handle (120) are cleaned and sterilized in while assembled.

The sponge (110) and handle (120) may be a disposable product not intended for reuse or reserialization. The sponge (110) may include a hydrophilic coating to help the sponge retain liquid. The hydrophilic coating may be over selected portions of the sponge or all of the sponge. The hydrophilic coating may be applied by dip coating. In one example, the hydrophilic coating is a functionalize silane coating.

The sponge (110) may be formed from a synthetic polymer. The sponge (110) may comprise a hydrophilic modification to enhance retention of aqueous solutions. The sponge (110) may be edible. For example, the sponge may be: polylactic acid (PLA), polyglycolic acid (PGA), hydrocolloids, polypeptides, polyvinyl acetate (PVA), polyethylene glycol (PEG), polypropylene glycol (PPG), and/or combinations thereof.

The device (100) may further include a reservoir. The reservoir may be connected to the handle (120). The reservoir may be capable of providing fluid to the sponge (110) through a lumen. The handle (120) may include a trigger or similar actuator to induce movement of fluid from the reservoir to the sponge. The fluid may move via capillary action from the reservoir to the sponge.

In one example, the handle (120) has a reduced cross sectional area on a distal end, the sponge (110) being located on the distal end of the handle (120). This may facilitate interaction with the patient mouth and/or reduce obstruction of the mouth. In one example, the distal end of the handle (120) includes blunted features to avoid injury and/or discomfort to the patient.

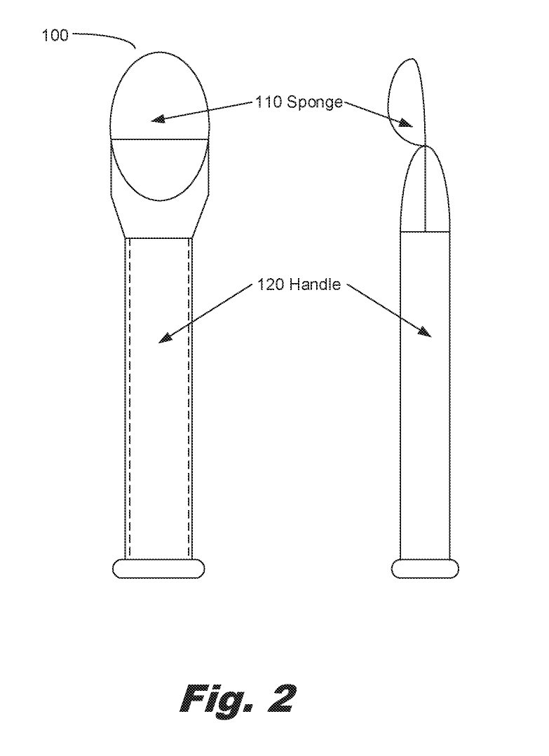

FIG. 2 shows a top view and a profile view of a device (100) according to one example consistent with the present disclosure. The device (100) includes a sponge (110) and a handle (120). FIG. 2 includes design elements that may make patient treatment easier and/or more reliable.

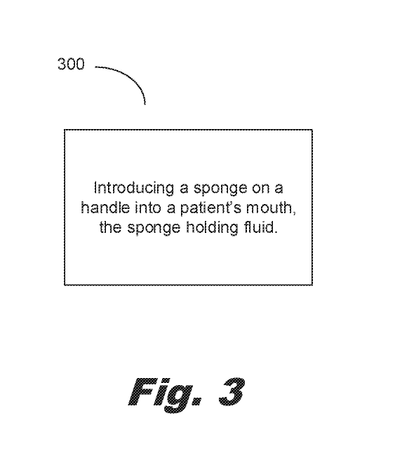

FIG. 3 outlines a method (300) consistent with the present disclosure. The method is a method (300) of providing oral hydration or feeding, the method (300) comprising introducing a sponge (110) on a handle (120) into a patient's mouth, the sponge holding liquid (310).

In some examples, the method (300) includes an additional operation performed before or after introducing a sponge (110) on a handle (120) into a patient's mouth, the sponge (110) holding liquid. The additional operation may include: contacting the sponge (110) with a liquid to absorb the liquid into the sponge (110); pressing the sponge (110) against a surface of the mouth to expel liquid from the sponge (110); and/or actuating a control on the handle (120) to provide liquid from a reservoir to the sponge (110).

The method (300) may include sterilizing the sponge (110). The method may include sterilizing the handle (120). The method (300) may include removing the sponge (110) and handle (120) from a sealed pouch. In one example the pouch is a heat sealed Tyvek™ pouch.

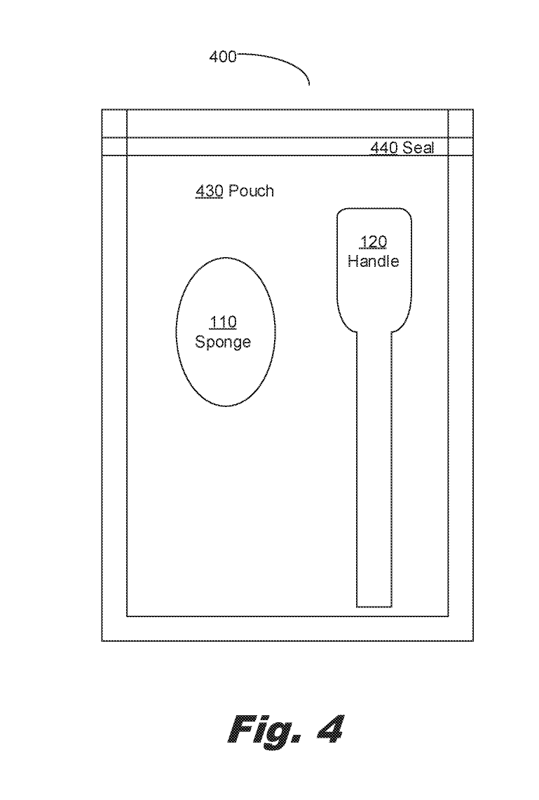

FIG. 4 shows a kit (400) according to one example consistent with the present specification. The kit (400) includes a sealed pouch (430) containing a sponge (110) and a handle (120). In one example, the sponge (110) and handle (120) are connected. In a second example, the sponge (110) and handle (120) are connectable.

The pouch (430) may be a Tyvek™ pouch. The pouch (430) may have a heat seal (440) on one side. The pouch (430) may multiple heat seals (440). The pouch (430) may be sealed mechanically, thermally, chemically, and/or using an adhesive.

The pouch (430) and its contents may be sterilized. Sterilization can be accomplished using steam, heat, e-beam, radiation, gamma, ethylene oxide, chemical sterilization, etc. In one example, the sealed pouch (430) is subjected to ethylene oxide sterilization as part of the manufacturing process. In another example, the sealed pouch (430) is subjected to radiation sterilization as part of manufacturing.

Within the principles described by this specification, a vast number of variations exist. The examples described are examples, and are not intended to limit the scope, applicability, or construction of the claims.

1. A device for providing fluid to a patient, the device comprising:
a food-contact-safe sponge;
a handle, the handle supporting the sponge,
a reservoir, and
an actuator, wherein each of multiple actuations of the actuator moves liquid from the reservoir to the sponge.

2. The device of claim 1, wherein the sponge comprises a synthetic polymer.

3. The device of claim 1, wherein the sponge further comprises a hydrophilic modification to enhance retention of aqueous solutions.

4. The device of claim 1, wherein the sponge is edible.

5. The device of claim 1, wherein the sponge comprises a material selected from a group consisting of: polylactic acid (PLA), polyglycolic acid (PGA) and mixtures thereof.

6. (canceled)

7. The device of claim 1, wherein the handle has a reduced cross sectional area on a distal end, the sponge being located on the distal end of the handle.

8. A method of providing oral hydration or feeding, the method comprising:
contacting the sponge with a liquid to absorb the liquid into the sponge;
introducing a sponge on a handle into a patient's mouth, the sponge holding liquid;
pressing the sponge against a surface of the mouth to expel liquid from the sponge, wherein the liquid provides treatment for hydration or feeding of the patient and
repeating contacting, introducing, and pressing the sponge to feed or hydrate the patient.

9-10. (canceled)

11. The method of claim 8, further comprising: actuating a control on the handle to provide liquid from a reservoir to the sponge.

12. A kit, the kit comprising:
a sealed, sterilized pouch containing:
a delivery sponge, the sponge to store liquid in an expanded state and release stored liquid upon compression; and
a handle to removably couple to the sponge, wherein handle supports the sponge and allows manipulation of the sponge.

13. (canceled)

14. The kit of claim 12, wherein a surface of the sponge is hydrophilic.

15. (canceled)

16. The device of claim 1, wherein most of the dry sponge consists of: polylactic acid (PLA), polyglycolic acid (PGA), hydrocolloids, polypeptides, polyvinyl acetate (PVA), polyethylene glycol (PEG), polypropylene glycol (PPG), and mixtures thereof.

17. The device of claim 12, wherein most of the sponge consists of: polylactic acid (PLA), polyglycolic acid (PGA), hydrocolloids, polypeptides, and mixtures thereof

18. The device of claim 12, wherein the pouch contains a plurality of delivery sponges and a single handle.

19. The device of claim 12, wherein the sponge further comprises a hydrophilic modification to enhance retention of aqueous solutions
